(19) United States
(12) Patent Application Publication
(10) Pub. No.: US 2017/0056658 A1
(43) Pub. Date: Mar. 2, 2017

(54) NEURAL SENSING DEVICE WITH INTERFERENCE SUPPRESSION

(71) Applicant: Po-Kang Lin, Taipei City (TW)

(72) Inventor: Po-Kang Lin, Taipei City (TW)

(21) Appl. No.: 14/840,077

(22) Filed: Aug. 31, 2015

(51) Int. Cl. A61N 1/36

(52) U.S. Cl. CPC A61N 1/36046

(57) ABSTRACT

The present invention provides a neural sensing device with interference suppression, including: an array of sensing units, a control signal generator and a signal processing module. The array of sensing units includes a plurality of sensing units. Each of the sensing units is configured to generate a sensing signal. The control signal generator is connected to the array of sensing units and is configured to generate a control signal to a surrounding of each of the sensing units. A signal strength of the control signal is lower than a signal strength of the sensing signals, so the control signal is isolated between the sensing signals between each of the sensing units, thereby suppressing interference. The signal processing module is connected to the array of sensing units and the control signal generator. The signal processing module generates and outputs a processing signal to at least one neuron.

### BACKGROUND OF THE INVENTION

1. Field of the Invention

The present invention relates to a neural sensing device, and more particularly, relates to a neural sensing device with interference suppression capable of avoiding interferences between the neurons caused by intensive signals.

2. The Prior Arts

In 2002, the Second Sight Medical Products Inc. performed human transplant operations on six blind patients utilizing its own product: the experimental electronic eye Argus I. Argus I is a bionic eye with a resolution of 16 pixels. With the differences in the brightness generated by the light reflection from the surfaces of the objects, Argus I gave blind patients the ability to distinguish the outlines of objects. In 2006, the company announced a second-generation product Argus II, which boosts the resolution up to 60 pixels. Argus II provides a better resolution and higher accuracy regarding the image recognition for the users.

The device of the experimental electronic eye Argus II includes an image capturing device disposed outside of the body of a user and an image receiving chip placed inside the body of the user. The image capturing device includes a frame, a miniature camera, an image processing unit and a wireless communication module. The frame is removably worn on the face of the user, and the miniature camera and the wireless communication module are equipped on the frame. The miniature camera is able to capture images in front of the user to generate an imaging information. In addition to being disposed on the frame, the image processing unit is also connected to the miniature camera. The image processing unit is able to receive the imaging information generated by the miniature camera and further generates a digital image information. At this time, the wireless communication module receives the digital image information generated by the image processing unit and transmits the digital image information to the image receiving chip. The image receiving chip is disposed on a retina of the user and is connected to at least one ganglion cell on the retina of the user. The image receiving chip is able to convert the digital image information into a neural spike, and is able to transmit the neural spike to the brain of the user via the at least one ganglion cell, thereby allowing the user to recognize an image.

In order to enhance the resolution of image and the recognition results perceived by the user, it is inevitable for the conventional image receiving chip to increase the number of photosensitive units within a limited area of the chip so as to increase the pixel value. By doing so, it would shorten the distance between each photosensitive unit, and the sensing signals of the photosensitive units are likely to interfere with each other during the operation. As a result, such configuration may have a pixel value that is even lower than the original effective pixel value. As shown in FIG. 1, when the sensing signals of two adjacent photosensitive units 10 are interfering with each other, and when the stacked signal between the two adjacent photosensitive units 10 is higher than a sensing threshold of at least one ganglion cell of the user, the two adjacent photosensitive units 10 will be recognized as one photosensitive unit 11 with a larger area by the at least one ganglion cell of the user, and the received sensing signals will be transmitted to the brain of the user. If all the photosensitive units are interfering with each other, the user will only be able to perceive a vague shape of light and shadow, thus losing the ability to identify images by the outline of the object. As a result, such image receiving chip with high pixels may lose its ability to enhance the resolution of image and recognition results for the users.

Therefore, there is an urgent need for the industry to develop a neural sensing device with interference suppression that may prevent the neurons from interfering with each other from intensive signals. It is preferred for such neural sensing device to have the characteristics of a small body, high neural sensing sensitivity and high accuracy.

### SUMMARY OF THE INVENTION

Based on the above reasons, a primary objective of the present invention is to provide a neural sensing device with interference suppression. Such neural sensing device is able to prevent the neurons from interfering with each other due to intensive signals, and further achieving the purpose of a device with high neural sensing sensitivity and high accuracy.

For achieving the foregoing objectives, the present invention provides a neural sensing device with interference suppression, including: an array of sensing units, a control signal generator and a signal processing module. The array of sensing units includes a plurality of sensing units. Each of the sensing units is configured to generate a sensing signal. The control signal generator is connected to the array of sensing units and is configured to generate a control signal to a surrounding of each of the sensing units. A signal strength of the control signal is lower than a signal strength of the sensing signals, so the control signal is isolated between the sensing signals between each of the sensing units, thereby suppressing interference. The signal processing module is connected to the array of sensing units and the control signal generator. The signal processing module generates and outputs a processing signal to at least one neuron. The sensing signal is higher than a sensing threshold of the at least one neuron, and the control signal is lower than the sensing threshold of the at least one neuron.

Preferably, the array of sensing units is an array of photodiodes of an electronic retina chip, and each of the sensing units is a photodiode for replacing a photoreceptor cell on a human retina.

Preferably, the control signal generator is disposed at a side of the array of the sensing units of an electronic retina chip, and the control signal generator transmits the control signal to the signal processing module.

Preferably, the neural sensing device can be directly connected to at least one ganglion cell on a human retina.

Preferably, the processing signal can be at least one spike.

When using the neural sensing device with interference suppression of the present invention, the control signal generator generates the control signal to a surrounding of each sensing units, so the control signal is isolated between the sensing signals between each of the sensing units. As a result, interferences can be suppressed, and the at least one neuron can be prevented from malfunction. When the array of sensing units senses image and/or light sources, each sensing unit on the array of sensing units generates the sensing signal according to the strength distribution of the light of the image and/or light source. Since the control signals are isolated between the each sensing signals as described above, when the array of sensing units senses the variation in the intensive distribution of the image and/or light source, the at least one neuron can be prevented from being interfered by multiple sensing signals. In this way, the present invention is able to provide a device with high neural sensing sensitivity and high accuracy.

### BRIEF DESCRIPTION OF THE DRAWINGS

FIG. 1 is a schematic view illustrating the interference between the sensing signals of two adjacent photosensitive units of the experimental electronic eye of the prior arts;

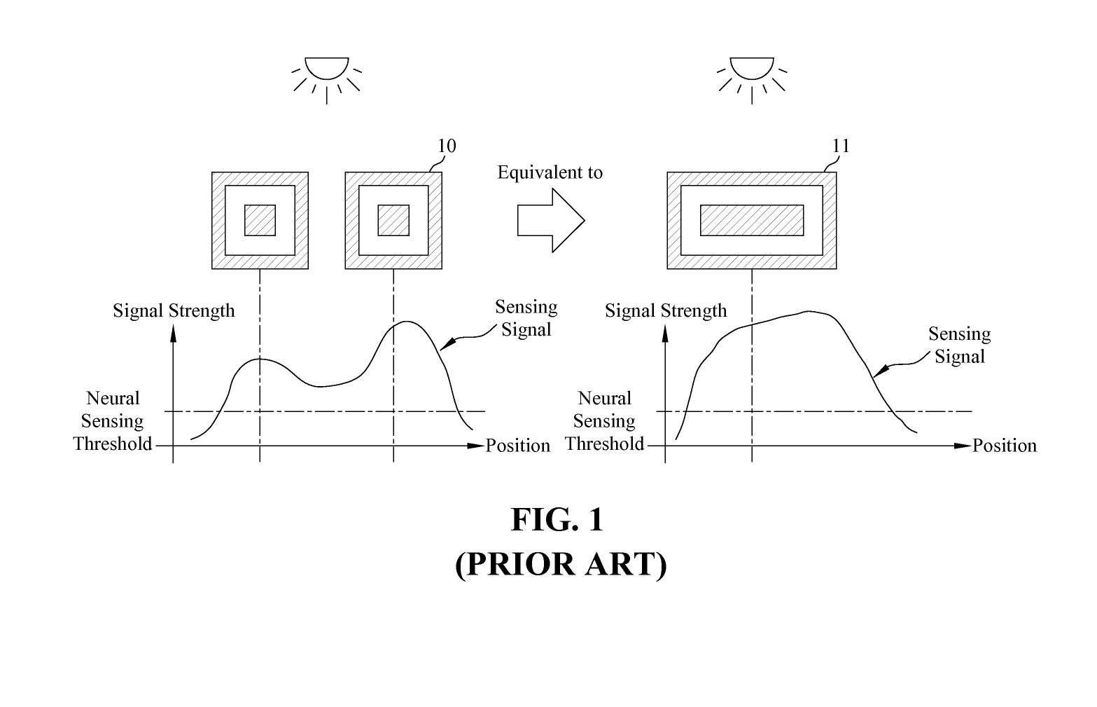

FIG. 2 is a block diagram schematically showing the circuits of a neural sensing device with interference suppression according to a first embodiment of the present invention;

FIG. 3 is a schematic view showing the appearance of an array of sensing units of the neural sensing device with interference suppression according to the first embodiment of the present invention;

FIG. 4 is a schematic view illustrating the coupling between a sensing signal and a control signal of the neural sensing device with interference suppression according to the first embodiment of the present invention; and

FIG. 5 is a schematic view illustrating the signal processing of the neural sensing device with interference suppression according to the first embodiment of the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention. The present invention may also be implemented or applied as other embodiments that are not described herein, and each details described in the specification may also be changed or modified according to different aspects without departing the spirit of the present invention.

It should be noted that the structure, ratio and size shown in the accompanying drawings of the present invention are only for illustrative purposes of the disclosure described in the specification, so those who skilled in the art may have a better understanding of the present invention. The structure, ratio and size shown in the accompanying drawings are not to limit the scope of the present invention; therefore, they do not represent any substantive technical meanings. Any structures modifications, ratios changes or size adjustment made to the drawings should still be considered to be within the scope of the present invention as long as they do not affect the effects and purposes thereof.

Hereafter, a neural sensing device with interference suppression will be described in accordance with a first embodiment of the present invention.

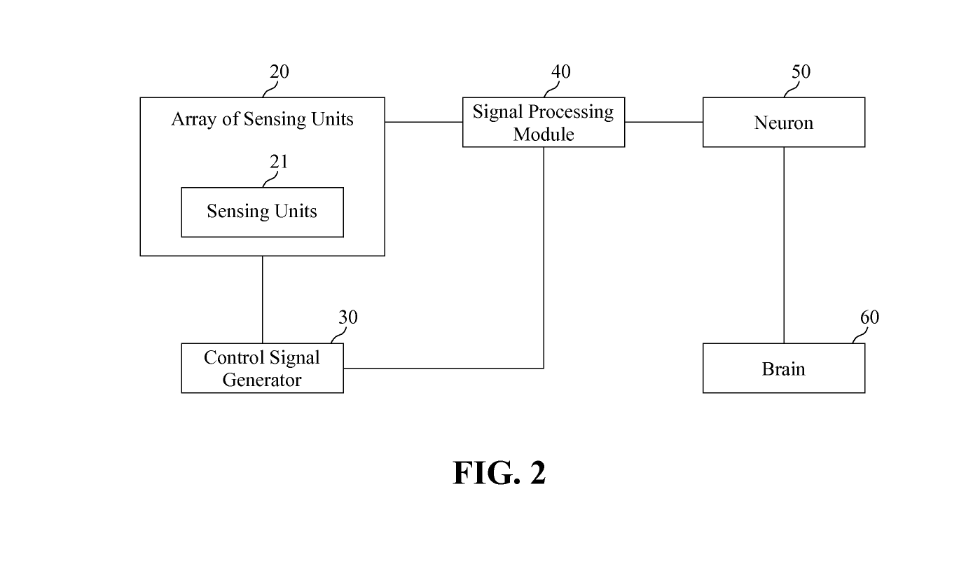

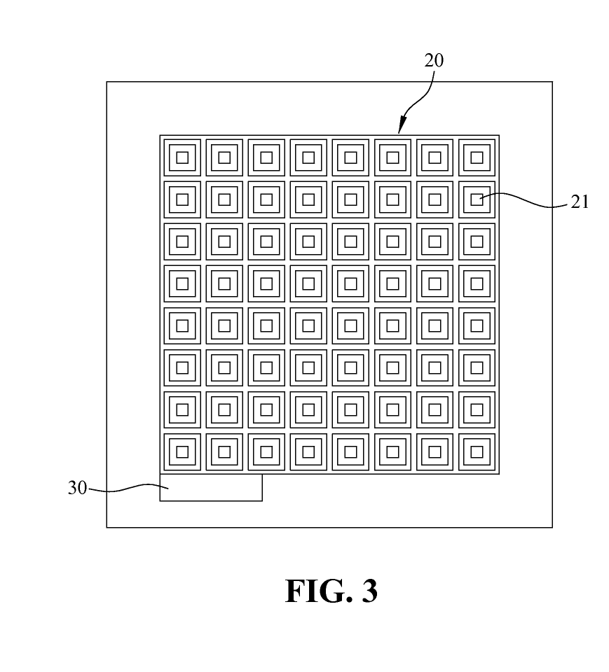

As shown in FIG. 2 and FIG. 3, the neural sensing device with interference suppression of the present invention includes: an array of sensing units 20, a control signal generator 30 and a signal processing module 40. The array of sensing units 20 is configured with a plurality of sensing units 21. Each of the sensing units 21 is configured to generate a sensing signal, and each sensing signals is higher than a sensing threshold of at least one neuron 50. In such a way, the at least one neuron 50 may sense effective information and transmit such information to the brain 60. According to the first embodiment of the present invention, the array of sensing units 20 is an array of photodiodes of an electronic retina chip. Each of the sensing units 21 is a photodiode that can be used to replace a photoreceptor cell on a human retina; namely, each of the sensing units 21 is treated as a pixel of an image to be sensed. Each sensing unit 21 is able to convert the photon energy emitted thereto into electronic ionization energy, and further output it as electric energy. In such a way, sensing signals corresponding to the photon energy can be generated, and the sensing signals can be transmitted to the signal processing module 40.

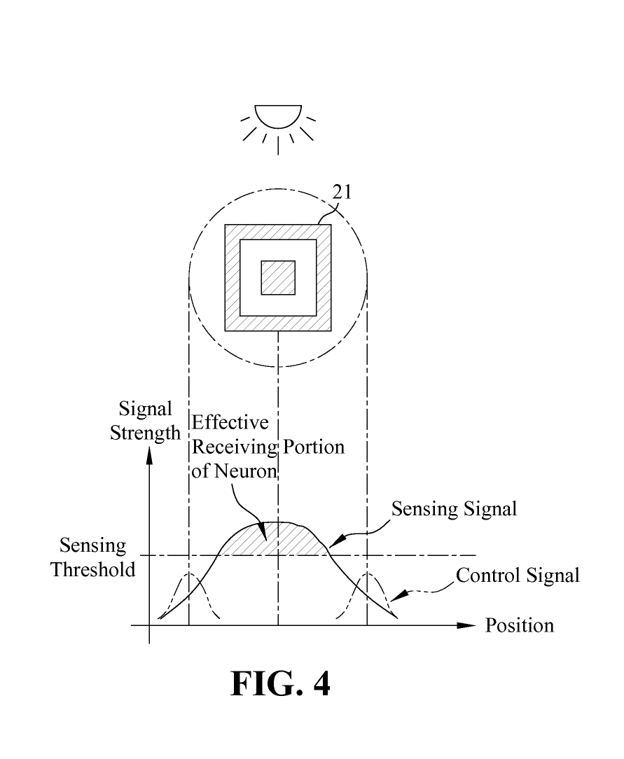

As shown in FIG. 2 to FIG. 4, the control signal generator 30 may be connected to the array of sensing units 20, and may be configured to generate a control signal to a surrounding of each sensing unit 21. The signal strength of the control signal is lower than the signal strength of the sensing signals, so the control signal is isolated between the sensing signals between each sensing units, thereby suppressing interference. Each control signal is lower than the sensing threshold of the at least one neuron 50 so as to prevent the at least one neuron 50 from sensing any effective information. According to the theory of neuron transmission, after the at least one neuron is action potential simulated, because of the inactivation of the sodium ion (N⁺) channel and a refractory period, the at least one neuron 50 cannot respond to other action potentials simulations when it is under the refractory period. The first embodiment of the present invention takes advantage of such a characteristic to put the at least one neuron 50 in the surrounding of each sensing unit 21 into the refractory period temporarily, and refrain the sensing signal generated by each sensing unit 21 from creating chain reactions at the surroundings thereof, thereby preventing the interferences between the sensing units. As a result, the situation in which the stacked signal strength between the adjacent sensing units 21 is higher than the sensing threshold of the at least one neuron 50 is prevented from happening. Furthermore, since the signal strength of each control signal never reaches the sensing threshold of the at least one neuron 50, effective information will never be constructed for the at least one neuron 50, thus the recognition results of the brain 60 toward the image to be sensed may stay unaffected. According to the first embodiment of the present invention, the control signal generator 30 may be configured at a side of the array of sensing units 20 of an electronic retina chip, and the control signal generator 30 may transmit the control signal to the signal processing module 40.

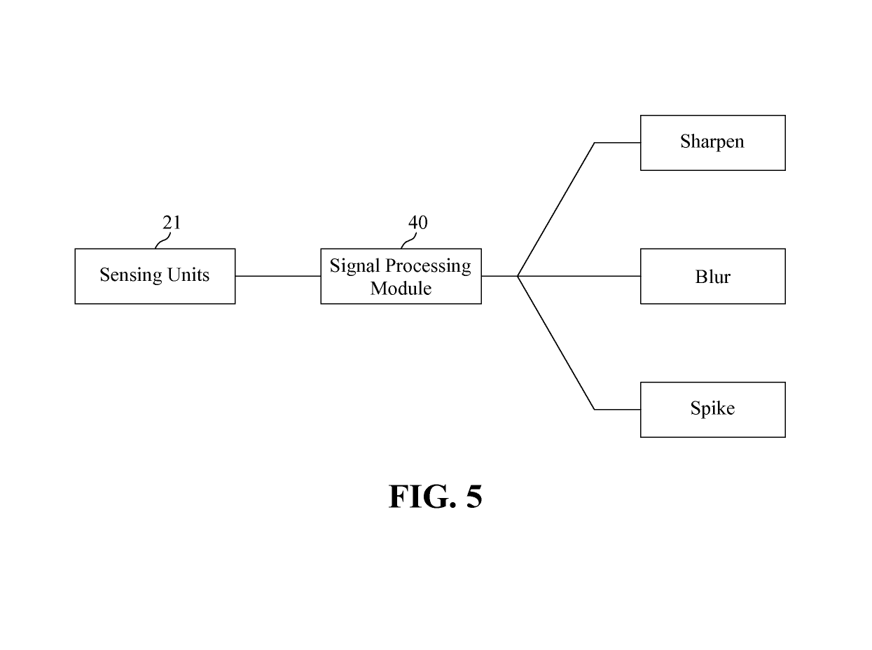

As shown in FIG. 2 and FIG. 5, the processing module 40 may be connected to the array of sensing units 20 and the control signal generator 30, and may be configured to generate and output a processing signal to the at least one neuron 50. According to the first embodiment of the present invention, neural sensing device may be directly connected to at least one ganglion cell on a human retina, so the signal processing module 40 may be used to replace the bipolar cell and/or horizontal cell on the human retina. Each of the at least one ganglion cells is a gangliform body structure formed by the congregation of the at least one neuron 50 of the same function. The bipolar cells are capable of enhancing the difference between the signal edges to increase the image sharpness perceived by the brain 60; that is, sharpening the image perceived by the brain 60. On the other hand, the horizontal cells are capable of reducing the difference in the signal edges to decrease the image sharpness perceived by the brain 60; that is, blurring the image perceived by the brain 60.

In the first embodiment of the present invention, the processing signal may be at least one spike. After the signal processing module 40 couples the sensing signals of each sensing unit 21 with the control signals, the signal process module 40 then generates and outputs the at least one spike to the at least one neuron 50.

When using the neural sensing device with interference suppression of the present invention, first, the array of sensing units 20 is installed on the retina of a user, and the at least one sensing unit 21 of the array of sensing units 20 is configured to be facing outward of the user to sense images outside of the retina of the user. The signal processing module 40 is then connected to the at least one ganglion cell on the human retina to transmit the processing signal to the at least one neuron 50. Subsequently, the control signal generator 30 generates the control signal to a surrounding of each sensing unit 21, so the control signal is isolated between the sensing signals between each sensing units; in such a way, interferences between the sensing signals can be suppressed, and the at least one neuron 50 can be prevented from malfunction. When the array of sensing units 20 senses image and/or light sources, each sensing unit 21 on the array of sensing units 20 generates the sensing signal according to the strength distribution of the light of the image and/or light source. Since the control signals are isolated between each sensing signals as described above, when the array of sensing units 20 senses variation in the intensive distribution of the light of the image and/or light source, the at least one neuron 50 can be prevented from being interfered by multiple sensing signals at the same time. In this way, the present invention is able to provide a device with high neural sensing sensitivity and high accuracy.

Although the present invention has been described with reference to the preferred embodiments thereof, it is apparent to those skilled in the art that a variety of modifications and changes may be made without departing from the scope of the present invention which is intended to be defined by the appended claims. In addition, the number of elements disclosed in the specification is only for illustrative purpose but to limit the scope of the present invention. The scope of the present invention should only be defined by the appended claims.

What is claimed is:

1. A neural sensing device with interference suppression, comprising:
an array of sensing units including a plurality of sensing units, wherein each of the sensing units is configured to generate a sensing signal;
a control signal generator connected to the array of sensing units and configured to generate a control signal to a surrounding of each of the sensing units, wherein a signal strength of the control signal is lower than a signal strength of the sensing signals, so the control signal is isolated between the sensing signals between each of the sensing units, thereby suppressing interference; and
a signal processing module connected to the array of sensing units and the control signal generator, wherein the signal processing module generates and outputs a processing signal to at least one neuron;
wherein the sensing signal is higher than a sensing threshold of the at least one neuron, and the control signal is lower than the sensing threshold of the at least one neuron.

2. The neural sensing device with interference suppression according to claim 1, wherein the array of sensing units is an array of photodiodes of an electronic retina chip, and each of the sensing units is a photodiode for replacing a photoreceptor cell on a human retina.

3. The neural sensing device with interference suppression according to claim 1, wherein the control signal generator is disposed at a side of the array of the sensing units of an electronic retina chip, and the control signal generator transmits the control signal to the signal processing module.

4. The neural sensing device with interference suppression according to claim 1, wherein the neural sensing device is directly connected to at least one ganglion cell of a human retina.

5. The neural sensing device with interference suppression according to claim 1, wherein the processing signal is at least one spike.
